(19) United States
(12) Patent Application Publication
(10) Pub. No.: US 2025/0072926 A1
(43) Pub. Date: Mar. 6, 2025

(54) PERICARDIAL TRANSECTION DEVICES AND METHOD

(71) Applicant: EDWARDS LIFESCIENCES CORPORATION, Irvine, CA (US)

(72) Inventors: Bezalel Haberman-Browns, Pardes Hanna (IL); Allen Jeong Keel, Irvine, CA (US); Mazen Saadi Albaghdadi, Irvine, CA (US); Gil Senesh, Adi (IL); Yehoshua Strauss, Elkana (IL); Gideon Meyer-Brodnitz, Ramot Menashe (IL); Michael Lis, Rishon Letzion (IL); Atiya Makhdoom Ahmad, Irvine, CA (US); Zachary Christopher Berwick, Tustin, CA (US); Tal Reich, Moledet (IL)

(21) Appl. No.: 18/954,086

(22) Filed: Nov. 20, 2024

Related U.S. Application Data
(63) Continuation of application No. PCT/US2023/023756, filed on May 26, 2023.
(60) Provisional application No. 63/347,524, filed on May 31, 2022.

(51) Int. Cl. A61B 17/32; A61B 17/00; A61B 17/3209; A61M 25/00

(52) U.S. Cl. CPC A61B 17/320016; A61B 17/3209; A61B 2017/00243; A61B 2017/00336; A61M 25/0068; A61M 25/0082

(57) ABSTRACT

Examples of transection devices are disclosed. One example includes an elongated body having a U-shaped notch, wherein the U-shaped notch includes a wall surface extending from a surface of the elongated body and an upper extension extending from the wall surface substantially parallel with the longitudinal axis of the elongated body. An incision member located in the U-shaped notch preferably extends from the elongated body toward the extension of the U-shaped notch at an angle. The incision member is well suited for cutting pericardial tissue as it passes through the U-shaped notch.

### RELATED APPLICATIONS

This application is a continuation application of International Patent Application No. PCT/US2023/023756, filed May 26, 2023, which claims the benefit of U.S. Provisional Application No. 63/347,524, filed on May 31, 2022. Each of the foregoing patent applications are hereby incorporated by reference in their entireties.

### TECHNICAL FIELD

This disclosure is directed to methods for treating heart failure, for example, heart failure with preserved ejection fraction (HFpEF) or reduced ejection fraction (HFrEF) by providing pericardial transection devices that introduce one or more incision lengths in a pericardium, e.g., a pericardial layer, fibrous layer, and/or adipose tissue.

### BACKGROUND

Pericardial restraint is a normal physiologic process that becomes exaggerated, for example, in some patients with heart failure with preserved ejection fraction (HFpEF) and causes the right heart to run out of space when filling, thereby squeezing and over pressurizing the left heart during physical activity in these patients. The increased left heart pressure backs up into the lungs and causes these patients to experience significant breathing difficulties when trying to do minimal activity, (exertional dyspnea). Exertional dyspnea is the most common symptom in patients with HFpEF and the most common cause for admission to the hospital in patients with HF in general. Currently, there is no therapeutic option for patients with HFpEF that specifically targets pericardial restraint.

### SUMMARY

In one example, a pericardial tissue transection device is provided, the device comprising: an elongated body with proximal end and a distal end, a longitudinal axis, a circumference, and a proximally-oriented U-shaped notch in the circumference, a portion of the U-shaped notch defining an extension parallel with the longitudinal axis; and an incision member extending from the elongated body to the extension, the incision member structured to receive and to cut pericardial tissue passing between the elongated body and the extension.

In one aspect, the elongated body is tubular. In another aspect, alone or in combination with any of the previous aspects, the elongated body is a flexible catheter with at least one lumen. In another aspect, alone or in combination with any of the previous aspects, the elongated body is a steerable catheter with at least one lumen. In another aspect, alone or in combination with any of the previous aspects, the elongated body is a multi-lumen catheter with at least one lumen. In another aspect, alone or in combination with any of the previous aspects, the elongated body has an outer diameter between about 6 Fr to about 30 Fr. In another aspect, alone or in combination with any of the previous aspects, the at least one lumen receives a guidewire.

In another aspect, alone or in combination with any of the previous aspects, the U-shaped notch having a wall surface separating the extension from the elongated body, the wall surface being continuous with the elongated body and the extension. In another aspect, alone or in combination with any of the previous aspects, the wall surface is substantially perpendicular to the longitudinal axis. In another aspect, alone or in combination with any of the previous aspects, the wall surface having a height of from 1 mm to 8 mm and a width smaller or equal to the circumference divided by pi.

In another aspect, alone or in combination with any of the previous aspects, the extension has a distal end and a proximal end integral with the elongated body. In another aspect, alone or in combination with any of the previous aspects, the distal end of the extension tapers to streamline with a guidewire. In another aspect, alone or in combination with any of the previous aspects, the distal end of the extension tapers towards the elongated body to a blunt tip.

In another aspect, alone or in combination with any of the previous aspects, the extension is separated from the elongated body by a distance of between 1 mm and 8 mm. In another aspect, alone or in combination with any of the previous aspects, the extension is uniformly separated from the elongated body by a distance of between 1 and 8 mm.

In another aspect, alone or in combination with any of the previous aspects, at least a portion of the elongated body comprises radiopaque material. In another aspect, alone or in combination with any of the previous aspects, at least a portion of the extension comprises radiopaque material.

In another aspect, alone or in combination with any of the previous aspects, the incision member extends from at least a portion of the wall surface. In another aspect, alone or in combination with any of the previous aspects, the incision member extends from the elongated body to the extension at an obtuse angle. In another aspect, alone or in combination with any of the previous aspects, the incision member extends from the elongated body to the extension at an acute angle. In another aspect, alone or in combination with any of the previous aspects, the incision member extends from the elongated body to the extension at a right angle.

In another aspect, alone or in combination with any of the previous aspects, the incision member is generally planar and extends from a center of the width of the wall surface, where at least a portion of the wall surface tapers away from at least one side of the incision member.

In another aspect, alone or in combination with any of the previous aspects, at least a portion of the incision member is a sharpened edge. In another aspect, alone or in combination with any of the previous aspects, at least a portion of the incision member is energizable with electrical current or radio frequency energy sufficient to separate pericardial tissue.

In another aspect, alone or in combination with any of the previous aspects, the incision member is a sharpened edge with at least a portion thereof energizable with electrical current or radio frequency energy sufficient to separate pericardial tissue.

In another aspect, alone or in combination with any of the previous aspects, the device further comprises a retractable sheath covering the extension. In another aspect, alone or in combination with any of the previous aspects, the device further comprises an atraumatic tip adjacent the distal end of the elongated body.

In another aspect, alone or in combination with any of the previous aspects, at least a portion of the retractable sheath comprises radiopaque material. In another aspect, alone or in combination with any of the previous aspects, the atraumatic tip receives a guidewire. In another aspect, alone or in combination with any of the previous aspects, at least a portion of the atraumatic tip comprises radiopaque material.

In another aspect, alone or in combination with any of the previous aspects, the device further comprises stabilizing members structured to reversibly extend laterally from the circumference of the elongated body. In another aspect, alone or in combination with any of the previous aspects, the stabilizing members are flexible rods, wires, or inflatables. In another aspect, alone or in combination with any of the previous aspects, the inflatables are balloons.

In another aspect, alone or in combination with any of the previous aspects, the device further comprises visualization means. In another aspect, alone or in combination with any of the previous aspects, the pericardial transection device is sterilized.

In another example, a pericardial tissue transection device is provided, the device comprising: an elongated body with proximal end and a distal end, and a longitudinal axis; a plurality of elongated members extending from a circumference of the distal end and coupled to a circumference of a coupling member, at least one of the plurality of elongated members comprising at least one incision portion; a first configuration where the plurality of elongated members extend substantially parallel with the longitudinal axis, and a second configuration where at least one of the plurality of elongated members comprising the at least one incision portion to extend laterally from the elongated body.

In one aspect, the device further comprises an inflatable member coupled to the plurality of elongated members to reversibly transition from the first configuration to the second configuration. In another aspect, alone or in combination with any of the previous aspects, the coupling member reversibly translatable from the distal end along the longitudinal axis causing the plurality of elongated members comprising the at least one incision portion to extend laterally.

In another aspect, alone or in combination with any of the previous aspects, the device further comprises an actuator member extending from a center of the distal end and coupled to a center of the coupling member such that translation of the actuator member translates the coupling member proximally to the distal end of the elongated body causing the at least one incision portion to extend laterally beyond an outer diameter of the elongated body, the incision member structured to receive and to cut pericardial tissue. at least one of the plurality of elongated members extend beyond an outer diameter of the elongated body and engage pericardial tissue. In another aspect, alone or in combination with any of the previous aspects, the plurality of elongated members comprises flexible rod or wire.

In another aspect, alone or in combination with any of the previous aspects, the incision portion extends laterally beyond an outer diameter of the elongated body and at least one of the plurality of elongated members extend beyond an outer diameter of the elongated body and can engage pericardial tissue providing stabilization of the elongated body.

In another aspect, alone or in combination with any of the previous aspects, the at least one incision member structured to receive and to cut pericardial tissue passing between the elongated body and the extension.

In another aspect, alone or in combination with any of the previous aspects, the at least one incision member is a conductive wire dielectrically coated with at least one skive portion through the dielectric coating exposing a portion of the conductive wire. In another aspect, alone or in combination with any of the previous aspects, the conductive wire comprises a plurality of spaced apart skive portions through the dielectric coating. In another aspect, alone or in combination with any of the previous aspects, the conductive wire comprises a continuous elongated skive portion through the dielectric coating.

In another aspect, alone or in combination with any of the previous aspects, the device further comprises a retractable sheath covering the plurality of elongated members.

In another aspect, alone or in combination with any of the previous aspects, at least a portion of the dielectrically coated conductive wire comprises radiopaque material. In another aspect, alone or in combination with any of the previous aspects, at least a portion of the retractable sheath or inflatable comprises radiopaque material. In another aspect, alone or in combination with any of the previous aspects, at least a portion of the coupling member comprises radiopaque material.

In another aspect, alone or in combination with any of the previous aspects, the device further comprises visualization means. In another aspect, alone or in combination with any of the previous aspects, the pericardial transection device is sterilized.

In another example, a method of incising pericardial tissue in a subject in need thereof is provided, the method comprising: providing a pericardial device of any one of the previous examples, introducing the pericardial device to a pericardial cavity; and incising at least a portion of a parietal layer of a pericardium along a length and a path.

In one aspect, the method further comprises reducing pericardial restraint of the subject. In another aspect, alone or in combination with any of the previous aspects, the pericardial device is introduced subxiphoidally. In another aspect, alone or in combination with any of the previous aspects, the pericardial device is introduced transvascularly. In another aspect, alone or in combination with any of the previous aspects, the pericardial device is introduced transvascularly via the Superior Vena Cava. In another aspect, alone or in combination with any of the previous aspects, the pericardial device is introduced transvascularly via the Inferior Vena Cava.

In another aspect, alone or in combination with any of the previous aspects, the incising of at least a portion of the parietal layer is by reverse cutting along a path and a length. In another aspect, alone or in combination with any of the previous aspects, the method further comprises repeating the step of incising the pericardial cavity along a different length, a different path, or a different length and a different path.

In another aspect, alone or in combination with any of the previous aspects, the method further comprises, after the introducing step and before the incising step, puncturing out of the pericardial cavity and exposing the incision portion. In another aspect, alone or in combination with any of the previous aspects, the method further comprises, after the of exposing the incision portion, receiving a least a portion of pericardial tissue in the U-shaped notch and presenting the pericardial tissue to the incision portion.

In a fourth example, a method of incising pericardial tissue in a subject in need thereof is provided, the method comprising providing a pericardial device of any one of the previous examples, introducing the pericardial device to a pericardial cavity, and incising at least a portion of a parietal layer of a pericardium along a length and a path.

In another example, a method of manipulating a medical device is provided, the method comprising: providing a medical device of any one of the previous claims engaged with a controller; and controlling at least one of movement of the blade between the retracted position and the deployed position, movement of the retractable cutting apparatus along the incision device, supplying energy to the incision device, or movement of one or more stabilizing members.

### BRIEF DESCRIPTION OF THE DRAWINGS

In order to understand and to see how the present disclosure may be carried out in practice, examples will now be described, by way of non-limiting examples only, with reference to the accompanying drawings, in which:

FIG. 1A is a sectional view of a 4-chambered heart.

FIG. 1B is an enlarged view of section 1B of FIG. 1A depicting the layers of the heart wall, including the pericardial cavity.

FIG. 1C is a further enlarged view of section 1C of FIG. 1A depicting the serosal, visceral, fibrous layers and adipose tissue of the parietal pericardium, including the pericardial cavity.

FIGS. 2A-2D depict a first example of a pericardial layer tissue transection device as disclosed or described herein.

FIGS. 3A-3E depict a second example of a pericardial layer tissue transection device as disclosed and described herein.

FIGS. 4A-4C depict a visualization system for use in a multi-lumen catheter device in combination with the presently disclosed pericardial layer tissue transection devices.

FIG. 5 depicts an exemplary controller device for delivering the presently disclosed transection devices, as disclosed and described herein.

FIG. 6 is a simplified diagram of a multi-lumen catheter approach to the pericardial cavity, as disclosed and described herein.

FIG. 7 is a simplified diagram of an alternative multi-lumen catheter approach to the pericardial cavity, as disclosed and described herein.

FIG. 8 is a simplified diagram of a parietal layer incision length and cut path as disclosed and described herein.

### DETAILED DESCRIPTION

Several exemplary devices for making incisions through the pericardial membrane or parietal layer of the pericardium are described. These examples share the characteristic that they are deployed intravascularly through the RAA, IVC, SVC, or via a subxiphoid approach.

As used herein the phrase “pericardial space” and pericardial cavity are used interchangeably and are inclusive of their ordinary and customary meaning to one of ordinary skill in medical and surgical arts, for example, a space, cavity, or liquid medium generally disposed between the parietal pericardium and visceral pericardium of a mammalian heart.

As used herein the phrase “pericardial tissue” is inclusive of its ordinary and customary meaning to one of ordinary skill in medical and surgical arts, for example, tissue associated with the pericardium.

As used herein, unless otherwise specified, the phrase “parietal layer” comprises at least the serosal and fibrous layer of the parietal pericardium, and optionally adipose tissue contained between, below, above, or within said layers. Further, the phrase “parietal layer” is inclusive of the ordinary and customary meaning to one of ordinary skill in medical and surgical arts, for example tissue layers generally disposed the adjacent to and including adipose tissue within and outside the pericardial cavity and superficial to the visceral layer of the pericardium.

As used herein the phrase “cutting surface” is inclusive of one or more of an edge of a sharpened blade or the surface of an electrode structured to receive sufficient current or radio frequency energy (RF) to ablate, burn, vaporize, or separate tissue. A cutting surface can be inclusive of both a sharpened edge and an electrode.

As used herein the phrase “reverse cutting” and “pull-back cutting” are used interchangeably and refer to methods involving the presentation of a cutting surface to tissue, the cutting surface adjacent a distal end of a multi-lumen catheter device or catheter, and the application of a directional force sufficient to cut or separate the tissue, the force being substantially in a direction towards the proximal end of the multi-lumen catheter device or catheter, for example, by pulling the multi-lumen catheter device or catheter while the cutting surface is engaged with the tissue.

It should be understood that the term “cutting” used herein refers to tissue disruption, for example, a sharp-cutting incision of the type associated with a knife blade such as a scalpel blade, or an electrosurgical device that provides electrical current to an electrically conductive material or electrode sufficient to disrupt tissue. The term “cutting” used herein includes “filet”, “slicing”, and the like.

As used herein the phrase “incision length” is inclusive of a non-zero distance of a cut or incision, for example, beginning at a first point, e.g., a target point, and terminating at a second point, e.g., an end point. An incision length can be linear, non-linear, or a plurality of linear and/or non-linear lengths that intersect or do not intersect about a curved or non-planar surface, such a heart.

As used herein the phrase “reducing pressure” and “reducing restraint” are inclusive of their ordinary and customary meaning of one to ordinary skill in medical and surgical arts.

As used herein the phrase “preserved ejection fraction” is inclusive of the ordinary and customary meaning to one of ordinary skill in medical and surgical arts, for example, a clinical syndrome in which patients display signs and symptoms of heart failure as the result of high left ventricular (LV) filling pressure despite normal or near normal left ventricle (LV) ejection fraction (LVEF; ≥50 percent).

As used herein the phrase “heart dysfunction” is inclusive of the ordinary and customary meaning to one of ordinary skill in medical and surgical arts, for example, heart failure or congestive heart failure.

As used herein the phrase “pericardial transection device” is inclusive of a device with an incision surface, for example an edge of a blade or a surface of an energized electrode.

As used herein the phrase “pericardial incision assembly” and “incision assembly” are used interchangeable and refer to an assemblage that includes a pericardial transection device.

As used herein the phrase “multi-lumen catheter device” is inclusive of a catheter structured with at least one lumen comprising a medical instrument, device, or component thereof, for example, a pericardial transection device.

As used herein, the terms “first,” “second,” and the like are only used to describe elements as they relate to one another, and are in no way meant to recite specific orientations of an article or apparatus, to indicate or imply necessary or required orientations of an article or apparatus, to indicate or imply necessary or required configurations of an article or apparatus, or to specify how an article or apparatus described herein will be used, deployed, transitioned from different configurations, or positioned in use.

As used herein, when an element is referred to as being “adjacent” and “coupled” when referring to two structures or layers, the two structures or layers are in proximity with one another with no intervening open space between them.

As used herein, when an element is referred to as being “coupled” or “adjacent” to another element, the two elements or structures are in proximity with one another, however, other elements or intervening elements may be present.

As used herein, when an element is referred to as being “directly coupled” or “directly adjacent” to another element, other elements or intervening elements are not present.

As used herein, term “operably coupled”, includes direct coupling and indirect coupling via another component, element, circuit, or structure and/or indirect coupling between items via an intervening item.

As used herein the phrase “nerve stimulation device” is inclusive of a device capable of applying an electrical potential to a nerve and to cause an observable effect that is directly or indirectly correlated to the applied potential, for example a pacing probe stimulating a phrenic nerve and causing an observable breathing disruption.

As used herein the phrase “nerve detecting device” is inclusive of a device capable of establishing a location or locale of at least part of a nerve and providing location or proximity information with no or substantially no physical effect or stimulus on the nerve, for example, an impedance sensor for detecting an electrical field generated by a nerve and to correlate, directly or indirectly, the location or proximity of the nerve relative to the impedance sensor.

As used herein the term “actuator” is inclusive of a mechanism for triggering an action.

As used herein the term “controller” is inclusive of a device having an actuator.

As used herein the phrase “biasing member” is inclusive of a device configurable in a stored energy state and a released energy state, for example, a spring.

As used herein the phrase “stabilizing member” is inclusive of a device configurable to impart stability and/or securement of a device to or within a structure, such as for example, stabilizing or securing a cutting surface positioned in a pericardial cavity from rolling, twisting, buckling and/or oscillating prior to or during use.

As used herein, “delta pulmonary capillary wedge pressure, Δ(PCWP)” is the difference between pulmonary capillary wedge pressure (PCWP) measured on volume challenge (i.e. leg raise and/or fluid administration) and PCWP at rest.

As used herein the phrase “puncturing tip” is inclusive of an atraumatic object suitable for puncturing or penetrating tissue without substantial trauma to or bleeding from the vicinity of the picture or penetration.

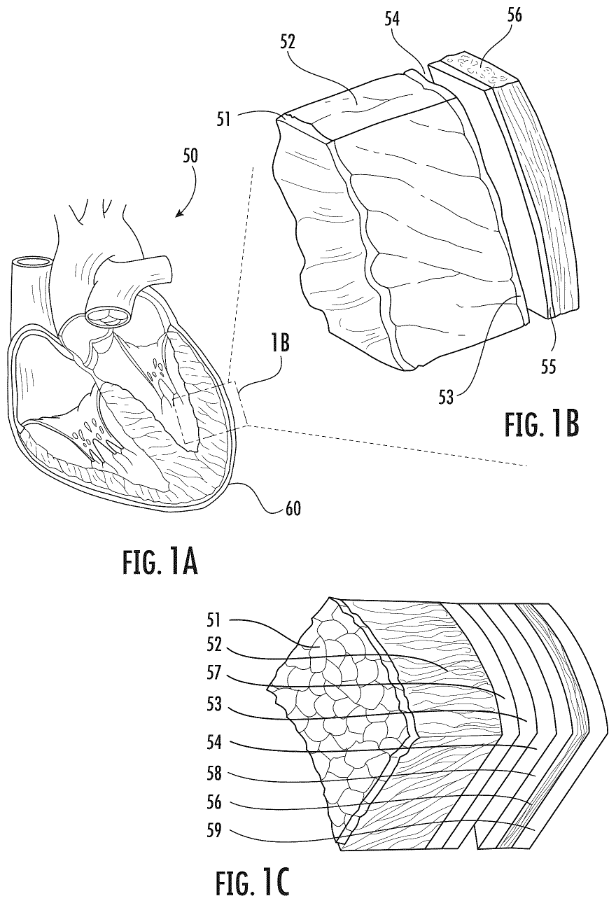

With reference to FIGS. 1A, 1B, 1C, and sections 1B, 1C, layers of a heart wall of a heart 50, from inside-out, being the endocardium 51, the myocardium 52, epicardial adipose tissue 57, the visceral layer 53 of the serous pericardium 55, the pericardial cavity 54, the parietal layer 58 of the serous pericardium 55, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity and superficial to the visceral layer 53 of the pericardium.

The presently disclosed pericardial tissue transection devices includes a perforating or puncturing portion designed to initially puncture the pericardial membrane. A guidewire, needle, microneedle knife, or electrical current may be used to form the perforation or puncturing of the pericardial membrane to allow access of the pericardial transection device to the pericardial cavity. Once the pericardial membrane is punctured, an incision assembly adjacent a distal end of a catheter or multi-lumen catheter is manipulated to a location within the pericardial cavity, and an incision member is allowed to engage with the pericardial tissue and an incision of a length along a path, for example, is created upon retraction of the pericardial tissue transection device towards the point of entry into the pericardial membrane. The incision member may alternatively, or in combination with a sharp edge, utilize RF energy to facilitate ease of incising and for providing some hemostasis or coagulation of incised edges of the pericardial membrane.

One or more incision of a length along a path can be made, either subxiphoidally, or transvascularly, by advancing an incision device, from the initial pericardial cavity entry location with cutting to a distal location (forward cutting). Alternatively, the incision length can be made by traversing the incision device from the initial pericardial cavity entry location to a distal location and then reversing back towards the entry location with cutting (reverse cutting). Thus, the presently disclosed pericardial transection devices are structured for either forward or reverse cutting modalities.

Several pericardial tissue transection device examples are shown in the attached figures. Hereinafter, the phrase “pericardial tissue transection device” and “transection device” shall be used interchangeably. Each transection device would be first introduced into the pericardial space via a transvascular or subxiphoid approach.

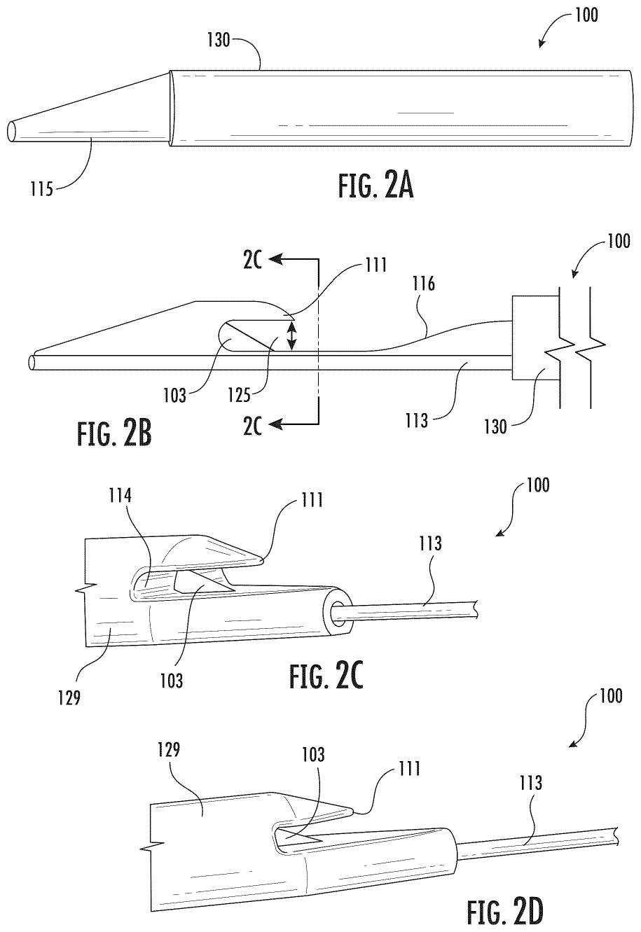

With reference to FIGS. 2A-2D, an exemplary pericardial tissue transection device 100 is shown. Device 100 has a “letter-opener” style configuration. Device 100 includes an atraumatic tip 115 designed to follow a guidewire after an initial puncture of the pericardial membrane. Atraumatic tip 115 can be structured to receive a guidewire 113 to facilitate introduction of the device to the pericardial membrane. Once the membrane is accessed, an incision member 103 of device 100 is maneuvered in the pericardial cavity 54 to a starting point of an incision path, and the incision member 103 engages the membrane and, upon retraction towards the point of entry into the pericardial space, creates an incision in the parietal layer in at least the parietal layer of a length and a long a path. In one example, the length and path of the incision is determined preoperatively. The blade may also utilize RF energy to facilitate ease of cut and hemostasis of the pericardial membrane.

Device 100 comprises an elongated body 129 (catheter) with proximal end and a distal end, a longitudinal axis, a circumference, and a U-shaped notch 125 within the circumference, a portion of the U-shaped notch defining an extension 111 parallel with the longitudinal axis. The U-shaped notch 125 outwardly tapers 116 to provide assistance with receiving pericardial tissue. An incision member 103 extends from the elongated body 129 to the extension 111, the incision member 103 structured to receive and to cut pericardial tissue passing between the elongated body and the extension. In one example, the elongated body 129 is tubular. In another example, the elongated body 129 is a flexible catheter with at least one lumen. The flexible catheter can be a steerable catheter or a steerable multi-lumen catheter with at least one lumen. An open end of the U-shaped notch 125 can be oriented proximally towards the proximal end of the elongate body 129. This orientation can facilitate reverse cutting of pericardium material.

In one example, the elongated body 129 comprises at least on lumen receiving a guidewire 113. In one example, the elongated body 129 has an outer diameter between about 6 Fr to about 30 Fr (about 2 mm to about 10 mm). In some examples, the elongated body 129 has an outer diameter between about 7 to about 12 Fr.

In one example, at least a portion of the elongated body comprises radiopaque material. As shown in FIG. 2C, depicting enlarged section 2C of FIG. 2B, U-shaped notch 125 comprises a wall surface 114 separating the extension 111 from the elongated body 129, the wall surface shown as being continuous with the elongated body and the extension.

Extension 111 has a distal end and a proximal end integral with the elongated body 129. As shown, the wall surface 114 is substantially perpendicular to the longitudinal axis of the elongated body 129. In one example, the wall surface 114 has a height of from 1 mm to 8 mm and a width equal to the circumference divided by the constant pi (π).

As shown, the distal end of the extension 111 tapers to a blunt tip so as to avoid damage to anatomy adjacent the pericardial membrane. The distal end of the extension 111 tapers towards the elongated body to a blunt tip. In one example, the extension 111 is separated from the elongated body 129 by a distance of between 1 mm and 8 mm. in one example, the extension 111 is uniformly separated from the elongated body 129 by a distance of between 1 and 8 mm.

At least a portion of the extension can comprise radiopaque material randomly dispersed or arranged in a pattern for visualization using conventional visualization techniques during use.

The incision member 103 of transection device 100 extends from at least a portion of the wall surface. As shown, the incision member 103 extends from the elongated body 129 to the extension at an obtuse angle relative to the longitudinal axis. In another example, the incision member 103 can extend from the elongated body 129 to the extension 111 at an acute angle or at a right angle.

The incision member 103 is generally planar in shape and as shown extends from a center of the width of the wall surface 114. In one example, at least a portion of the wall surface tapers away from at least one side of the incision member 103 to facilitate and assist separation of the pericardial tissue that is received and cut by the incision member positioned in the U-shaped notch 125.

In one example, at least a portion of the incision member 103 is a sharpened edge or is energizable with electrical current or radio frequency energy sufficient to separate pericardial tissue. In another example, the incision member 103 is a sharpened edge with at least a portion thereof energizable with electrical current or radio frequency energy sufficient to separate pericardial tissue.

Transection device 100 is shown comprising a retractable sheath 130 structured to reversibly cover the extension 111 and the incision member 103 during manipulation through the pericardial cavity 54. Retractable sheath 130 is structured to be withdrawn to expose the U-shaped notch and incision member 103 and to receive and to cut pericardial tissue. At least a portion of the retractable sheath 130 can comprise radiopaque material randomly distributed or arranged in a pattern for visualization using conventional visualization techniques during use.

Transection device 100 is shown further comprising an atraumatic tip 115 adjacent the distal end of the elongated body 129. Atraumatic tip 115 is structured to receive a guidewire 113. At least a portion of the introducer/dilator can comprise radiopaque material randomly distributed or arranged in a pattern for visualization using conventional visualization techniques during use.

Transection device 100 can further comprise stabilizing members 120 (not shown) structured to reversibly extend laterally from the circumference of the elongated body 129 as further described below for alternative transection devices. Such stabilizing members 120 can be flexible rods, wires, or inflatables, such as balloons. Transection device 100 can further comprise visualization means as further described below. Transection device 100 is structured for sterilization using conventional techniques such as ethylene oxide, electron beam, gamma, and autoclaving as well as chemical sterilization and aseptic manufacturing and/or packaging techniques.

Transection device 100 is structured for sterilization using conventional techniques such as ethylene oxide, electron beam, gamma, and autoclaving as well as chemical sterilization and aseptic packaging techniques.

In a second example, a mechanical or an expandable balloon-type radiofrequency (RF) transection device 300 is employed. The transection device 300 is deployed into the pericardial space and inflated or otherwise mechanically extended laterally from an elongated body 129. Upon lateral extension, and incision region is pressed against the pericardial membrane. The incision region can consist of a cutting blade, or an RF energized cutting wire, or combination of a cutting blade and an RF energized wire. In one example, a predefined length of cut is made before the balloon is deflated or the lateral extension is mechanically reversed and repositioned for a second cut to extend the first cut, for example. The resultant incision in the pericardial membrane can be lengthened by repeating this process. Alternatively, the cutting element and mechanical/inflation lateral extension may be sufficiently long enough to cover the entire length of the intended incision, thereby enabling a single cut method to achieve the desired outcome.

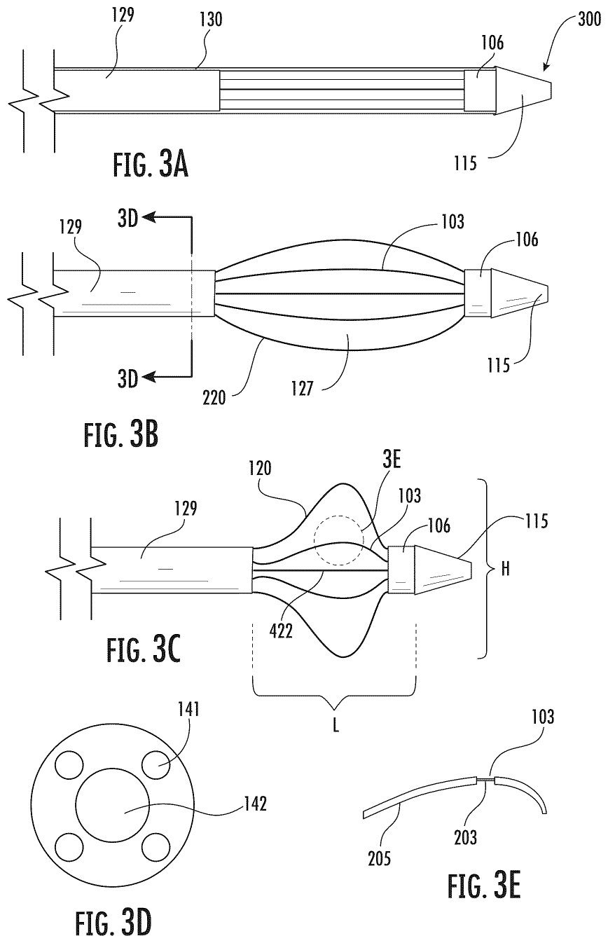

An example of such an RF energized transection device 300 is shown in FIGS. 3A-3E, where transection device 300 comprises an elongated body 129 with proximal end and a distal end, and a longitudinal axis and a plurality of elongated members extending from a circumference of the distal end and coupled to a circumference of a coupling member 106, at least one of the plurality of elongated members comprising at least one incision member 103, transection device 300 having a first configuration where the plurality of elongated members extend substantially parallel with the longitudinal axis, and a second configuration where at least one of the plurality of elongated members comprising the at least one incision portion to extend laterally from the elongated body. As shown in section view along line 3D-3D, FIG. 3D depicts a multi-lumen catheter device with a plurality of lumens 141, 142 to receive various elements and members for the transection devices, incision assembly, stabilizing members, to provide contrast fluid, electrical current and the like.

In one example, transection device 300 further comprises an inflatable member 127 coupled to the plurality of elongated members to reversibly transition from the first configuration to the second configuration, where in the second configuration the at least one incision member 103 receives and cuts pericardial tissue. In another example, transection device 300 further comprises an actuator member 422 extending from a center of the distal end of the elongated body 129 and is coupled to a center of the coupling member 106 such that translation of the actuator member 422 translates the coupling member 106 from an initial length L proximally to the distal end of the elongated body 129 causing the at least one incision member 103 to extend laterally a distance H, for example, beyond an outer diameter of the elongated body 129, the incision member structured to receive and to cut pericardial tissue.

In one example, the plurality of elongated members 220 is a flexible rod or wire. The incision member 103 extends laterally beyond an outer diameter of the elongated body 129 and at least one of the plurality of elongated members 220 extends beyond an outer diameter of the elongated body and engage pericardial tissue providing stabilization of the elongated body and the incision portion so as to receive and to cut pericardial tissue passing between the elongated body and the extension.

As shown in FIG. 3E, the enlarged view of section 3E of FIG. 3D, the at least one incision member 103 is a conductive wire dielectrically coated with at least one skive portion 203 through the dielectric coating 205 exposing a portion of the conductive wire as the incision member 103.

In one example, the conductive wire comprises a plurality of spaced apart skive portions 203 through the dielectric coating 205. In yet another example, the conductive wire comprises a continuous elongated skive portion 203 through the dielectric coating 205.

At least a portion of the dielectrically coated conductive wire can comprise radiopaque material randomly distributed or arranged in a pattern for visualization using conventional visualization techniques during use.

Transection device 300 can further comprise a retractable sheath 130 covering the plurality of elongated members 220. At least a portion of the retractable sheath 130 can comprises radiopaque material randomly distributed or arranged in a pattern for visualization using conventional visualization techniques during use.

Transection device 300 can further comprise visualization means as further described below. Transection device 300 is structured for sterilization using conventional techniques such as ethylene oxide, electron beam, gamma, and autoclaving as well as chemical sterilization and aseptic packaging techniques.

The RF wire can be selectively insulated along various lengths so that the insulated, or non-cutting, areas maintain contact and position within the pericardial space. Furthermore, several RF wires may be used, for example two, three, four, or more, in which a number of the wires fix the cutting device into position and one (or more) of the wires that contact the pericardial membrane are activated to make the desired cut. Orientation of the “basket” of wires as shown in FIG. 3C can be achieved using imaging in combination with radiopaque marking of selected wires, for example. This method can be further adapted to provide a specific geometry or pattern of cut as desired to achieve optimal reductions in the intracardiac pressures.

In one example, a puncture to deliver a guidewire into the pericardial space is performed through heart tissue. When a transvascular approach through the RAA, IVC, or SVC is employed, a closure device may be subsequently introduced for hemostasis at the conclusion of the procedure. In one example, the closure device includes outward or radially directed splines deployed in an expanded configuration. When the guide catheter is removed, the splines or radial members of the closure device contract inwardly towards the unstressed state of the transection device in order to close, occlude, and/or seal the opening. The closure device is designed such that a pericardial cutting device can pass through and into the pericardial space.

The following exemplary occlusion descriptions relate to a transvascular approach through the RAA, IVC, or SVC using one of the aforementioned transection devices 100, 300. In one example, an atraumatic tip 115 delivers a wire into the pericardial space through heart tissue. A closure or occlusion device is introduced for hemostasis during the procedure. The closure or occlusion device in one example includes outward or radially directed splines deployed in an expanded configuration. When the guide catheter is removed, the splines or radial members of the closure device contract inwardly towards the unstressed state of the transection device in order to close and seal the opening. The closure device is designed such that a pericardial cutting device can pass through and into the pericardial space.

With reference to FIGS. 1A, 1B, 1C, and sections 1B, 1C, layers of a heart wall of a heart 50, with pericardium 60, from inside-out, being the endocardium 51, the myocardium 52, epicardial adipose tissue 57, the visceral layer of the serous pericardium 53, the pericardial cavity 54, the parietal layer of the serous pericardium 55, and the fibrous pericardium 56, and pericardial adipose tissue 59 are depicted. In one example, the presently disclosed devices are structured for introduction to the pericardial cavity 54 and for cutting tissue layers generally disposed adjacent to and including adipose tissue within and outside the pericardial cavity 54 and superficial to the visceral layer 53 of the pericardium 60.

In one example, to provide orientational stability of the cutting surface to that of the parietal layer, an OTW introduction is employed for any of the previously disclosed devices, for example, whether through a dedicated lumen in multi-lumen catheter cross-section or ‘Rapid Exchange’ style catheter, or off-center attached cannula, or deflect-resistant catheter, as the delivering catheter randomly distributed or arranged in a pattern for visualization using conventional visualization techniques during use.

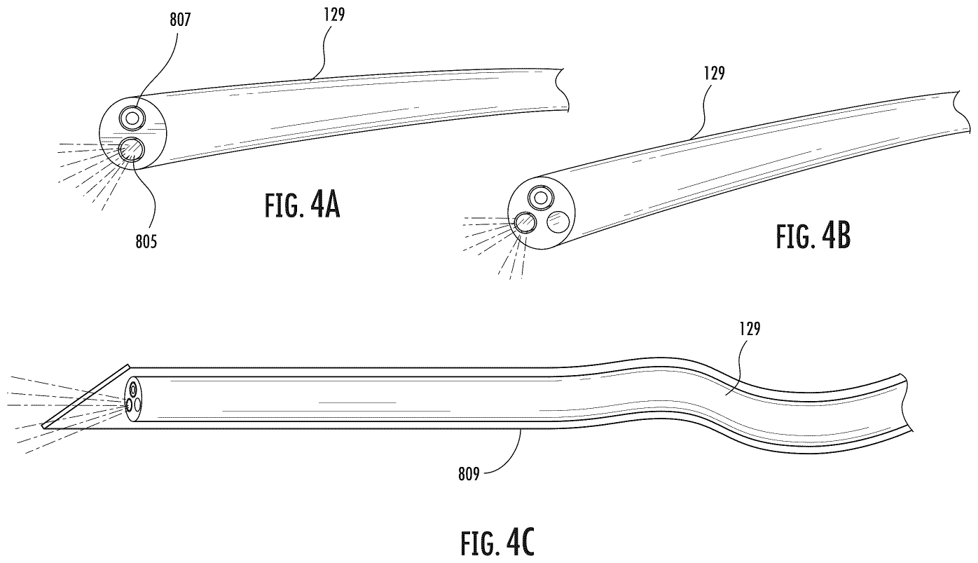

Current ECHO/fluoroscopy may not provide the required visualization for certain access applications of the presently disclosed transection devices, for example, gaining guidewire access pericardial cavity consistently and repeatedly may be desired. Thus, in one example, the multi-lumen catheter device 129 coupled to the presently disclosed transection devices comprises direct visualization, as shown in FIGS. 4A-4C, allowing the user to watch real-time the advancement of the transection device 100, 300 through various tissue layers until the desired location is reached. Changes in tissue layers that may not be visible under ECHO/fluoroscopy may be easily distinguishable under direct visualization such as tissue (vessel access), myocardium/pericardium (pericardial cavity access), myocardium/pericardium (outside pericardium), among other anatomical features.

In one example, the presently disclosed devices discussed above further comprise an optical channel in the multi-lumen catheter to accommodate a lens coupled to a fiber optic cable, optionally with a light source, e.g., an LED. In one example, the presently disclosed method further comprises obtaining visual information during accessing, traversal of the pericardial cavity, exiting and/or cutting, for example, using an optical channel in the multi-lumen catheter to accommodate a lens coupled to a fiber optic cable, optionally with a light source, e.g., an LED.

As shown in FIGS. 4A-4C, multi-lumen catheter 129, shown without a transection device for clarity, comprises a fiber optic channel and lens 807 adjacent a fiber optic channel 805 to provide light and to provide an analog or digital image in a multi-lumen catheter 129, which may also have a sheath 809.

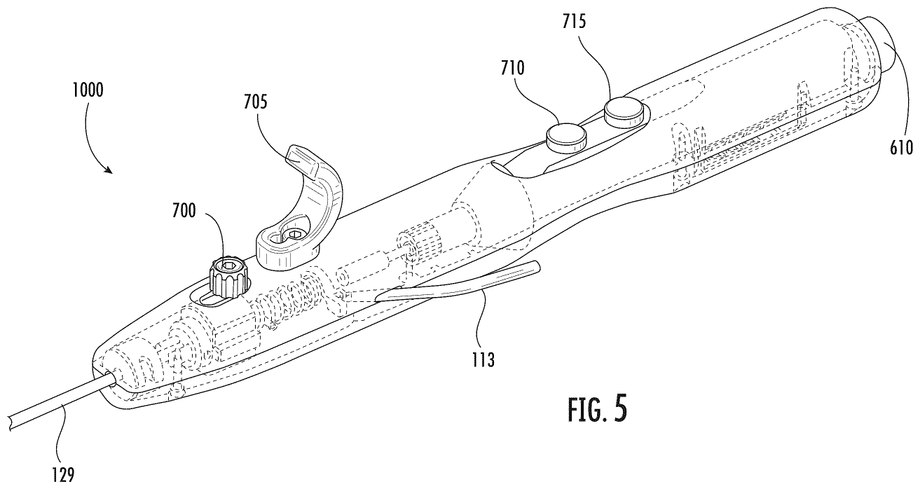

With reference to FIG. 5, transection devices 100, 300, can be manipulated and/or controlled from outside the subject using a controller 1000, which may be a handle. The controller 1000 may have multiple actuating knobs 700, 705, 610 and actuating buttons 710, 715 for controlling the multi-lumen catheter 129 and the various components of the transection device 100, 200, 300. Knob 700 may be structured to deploy stabilizing members 120 and/or rotate the flexible catheter 129 in response to orientation information derived from fluoroscopy or other visualization means. Knob 705 may be engaged to activate one or more components on the medical device (e.g., actuating the stabilizing member(s)). Similarly the actuating buttons 710, 715 may be engaged to activate various components of the multi-lumen catheter (e.g., the device may use RF electrode cutting as well and the actuating buttons 710, 715 may be used to supply current or RF). Various other controllers are envisioned that allow for the aforementioned transection devices to be deployed and manipulated.

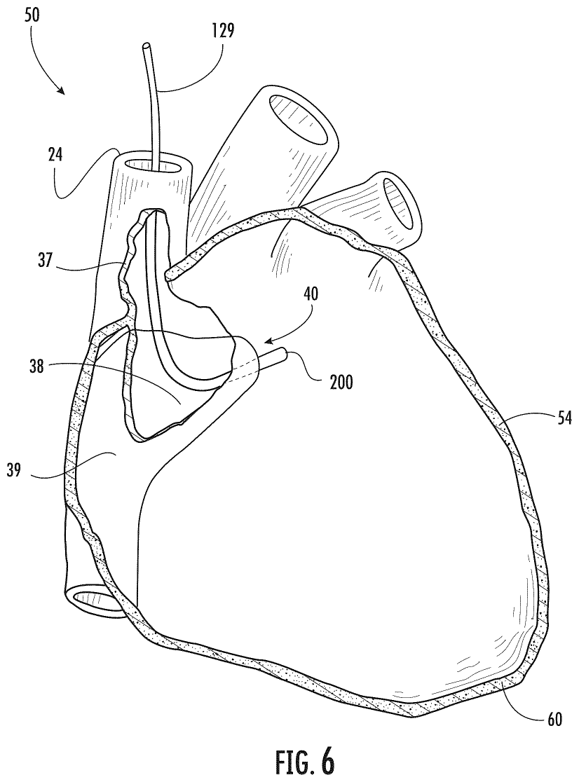

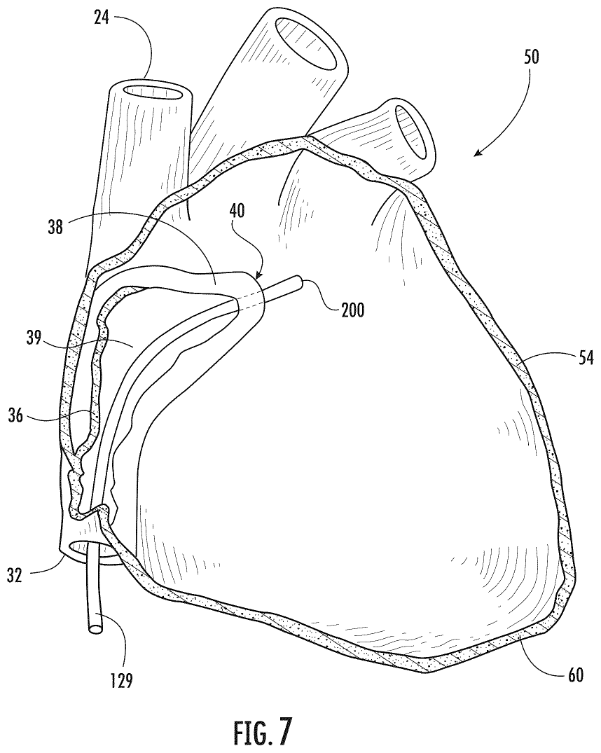

FIGS. 6 and 7 shows various intravascular approaches for delivering the transection devices of the present disclosure to the pericardial cavity 54. Thus, FIG. 7 depicts heart 50 viewed in isolation from the body, with the pericardium 60 or pericardial sac encasing the cardiac muscle (i.e., epicardium, myocardium, and endocardium). The small space which is present between the heart muscle and pericardium 60 represents the pericardial cavity 54.

The presently disclosed transection devices that can be presented to the pericardial cavity 54. In one example via the right atrial appendage 38 (RAA), which is a suitable site for entry into the pericardial cavity 54, is used. Right atrial appendage 38 lies tangential to and between pericardium 60 and the epicardium/epicardial adipose tissue 57. In one example, any of the presently disclosed devices is guided into right atrial appendage 38 via right atrium 39 so as to be positioned substantially in parallel with the wall of pericardium 60 such that when the wall of right atrial appendage 38 is pierced by the transection device 100, 200, 300 it is done substantially without risk of damaging the epicardium or other heart tissue. Other access routes to the pericardial cavity can be used, for example, direct “puncture out” of SVC or IVC or coronary sinus (CS) and a “puncture into” the pericardium.

In some examples, right atrial appendage 38 may be accessed via conventional vena cava routes. FIG. 6 illustrates entry of any of the presently disclosed devices into right atrium 39 via the superior vena cava 24 (SVC). A cut-away 37 shows passage of any of the presently disclosed devices through superior vena cava 24, right atrium 39, and right atrial appendage 38. A distal tip of catheter 129 is shown exiting right atrium 39 at tip 40.

FIG. 7 illustrates an alternative entry of any of the previously disclosed devices into right atrium 39 via the inferior vena cava 32 (IVC). A cut-away 36 shows passage of catheter 129 through inferior vena cava 32, right atrium 39, and right atrial appendage 38. A distal tip of catheter 129 is shown exiting right atrium 39 at tip 40.

Thus, by way of example, the method of the present disclosure includes the following steps. Any of the presently disclosed devices is maneuvered through one of the vena cava 24, 32 to right atrium 39. Once inside right atrium 39, any of the presently disclosed devices is passed into the right atrial appendage 38. The wall of right atrial appendage 38 is pierced at tip 40, and the catheter is advanced into the pericardial cavity 54. Other transvascular-right heart routes to the pericardial cavity 54 are envisaged.

Note that the wall of the right atrial appendage may be pierced with any of the presently disclosed devices itself, or with an instrument (e.g., guidewire) passed through a lumen of the any of the presently disclosed devices, e.g., over the wire. Further, any of the previously disclosed devices may be passed into the pericardial space through the opening in the wall of the atrial appendage, or an instrument passed through the lumen of any of the presently disclosed devices may be presented into the pericardial cavity 54. These details will depend on the procedure being performed and on the type of the previously disclosed device being employed.

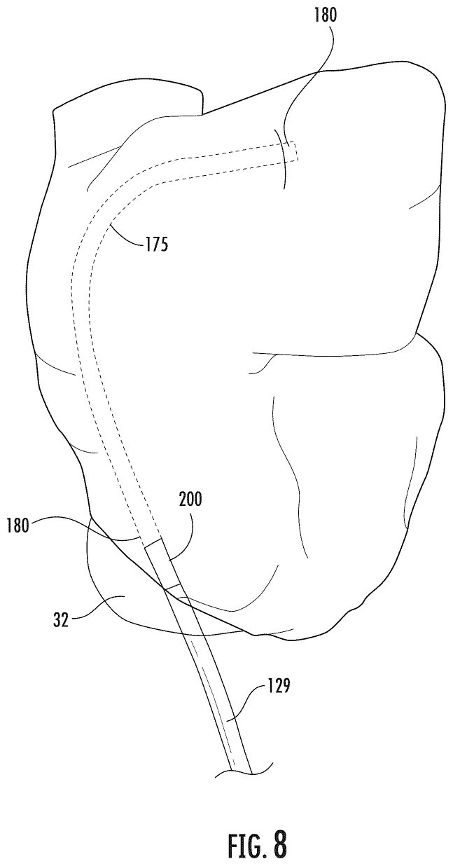

As shown in FIG. 8, a steerable catheter 129 can be employed, extending through the IVC, through the RA, and into the RAA and then into the pericardial cavity 54, the steerable catheter having a plurality of steerable segments. In some examples, the steerable catheter guiding the transection device 100, 200 300 can have a radius of curvature of between about 1 inches and about 5 inches, with an arc length of between about 90° and about 180°. As shown in FIG. 8, device 100, 200, 300 begins a cut path 175 at a start point 160 and ends at endpoint 180. At least a portion of the parietal layer of the serous pericardium 55, and the fibrous pericardium 56, and pericardial adipose tissue 59 are separated along cut path 175. The one or more incisions along the lengths, in a heart with a dysfunction treatable with the present method, cause the pericardium to separate radially about the cut line of the incision path 175, without the removal of pericardial tissue. One or more cut paths 175 can be made, and different cut paths, of various lengths can be used to reduce pericardial restraint. In one example, the cut path 175 and its length is pre-operatively determined. Other cut paths and lengths can be used.

In one example, the presently disclosed device further comprises at least one nerve detection device. In one example, the at least one nerve detection device is located on the flexible catheter 129. In one example, the at least one nerve detection device is located adjacent the incision assembly. In one example, the at least one nerve detection device is located on the atraumatic tip 115. In one example, the at least one nerve detection device is located on the incision member 103.

Any one of the presently disclosed devices can further comprises at least one nerve stimulation device. In one example, the at least one nerve stimulation device is located on the flexible catheter 129. In one example, the at least one nerve stimulation device is located adjacent the incision assembly. In one example, the at least one nerve stimulation device is located on introducer/dilator 115. In one example, the at least one nerve stimulation device is located adjacent the incision member 103.

The presently disclosed pericardial transection devices and/or catheter and/or sheath can be structured such that the total outer diameter (O.D.) introduced to the pericardial cavity is between about 6 Fr (2 mm) and about 30 Fr (10 mm). In one example, presently disclosed pericardial transection devices and/or catheter and/or sheath can be structured such that the total outer diameter (O.D.) introduced to the pericardial cavity is between about 6 Fr (2 mm) and about 20 Fr (6.67 mm). The pericardial transection device and/or catheter and/or sheath can be structured such that the total outer diameter (O.D.) introduced to the pericardial cavity is between about 6 Fr (2 mm) and about 15 Fr (5 mm). In one example, presently disclosed pericardial transection devices and/or catheter and/or sheath can be structured such that the total outer diameter (O.D.) introduced to the pericardial cavity is between about 6 Fr (2 mm) and about 12 Fr (4 mm). In one example, presently disclosed pericardial transection devices and/or catheter and/or sheath can be structured such that the total outer diameter (O.D.) introduced to the pericardial cavity is approximately 10 Fr (3.33 mm).

A kit, comprising any one of the presently disclosed medical devices, a sheath 130, a guidewire 113, and an introducer/dilator is provided.

While certain examples of the present disclosure have been illustrated with reference to specific combinations of elements, various other combinations may also be provided without departing from the teachings of the present disclosure. Thus, the present disclosure should not be construed as being limited to the particular exemplary examples described herein and illustrated in the Figures but may also encompass combinations of elements of the various illustrated examples and aspects thereof.

We claim:

1. A pericardial tissue transection device comprising:
an elongated body extending along a longitudinal axis between proximal and distal ends, the elongated body comprising a U-shaped notch defined in the elongated body, wherein the U-shaped notch comprises a wall surface extending from a surface of the elongated body and an upper extension extending from the wall surface substantially parallel with the longitudinal axis of the elongated body; and
an incision member located in the U-shaped notch and extending from the elongated body toward the extension of the U-shaped notch at an angle, wherein the incision member is structured to receive and to cut pericardial tissue passing through the U-shaped notch.

2. The pericardial tissue transection device of claim 1, wherein the elongated body has an outer diameter between about 6 Fr to about 30 Fr.

3. The pericardial tissue transection device of claim 1, wherein the wall surface extends substantially perpendicular to the longitudinal axis of the elongated body.

4. The pericardial tissue transection device of claim 1, wherein the wall surface extends from the surface of the elongated body to a height of from 1 mm to 8 mm and has a width smaller or equal to a circumference of the elongated body divided by pi, and wherein the extension is separated from the surface of the elongated body by a distance of between 1 mm and 8 mm.

5. The pericardial tissue transection device of claim 1, wherein the incision member extends from at least a portion of the wall surface of the U-shaped notch.

6. The pericardial tissue transection device of claim 1, wherein the incision member extends from the elongated body toward the extension at an obtuse angle, at an acute angle, or at a right angle.

7. The pericardial tissue transection device of claim 1, wherein the incision member is generally planar and extends from a center of a width of the wall surface, where at least a portion of the wall surface tapers away from at least one side of the incision member.

8. The pericardial tissue transection device of claim 1, wherein at least a portion of the incision member is a sharpened edge or wherein at least a portion of the incision member is energizable with electrical current or radio frequency energy.

9. The pericardial tissue transection device of claim 1, further comprising a retractable sheath covering the extension.

10. The pericardial tissue transection device of claim 1, further comprising stabilizing members structured to reversibly extend laterally from the elongated body, wherein the stabilizing members are one or more of flexible rods, wires, or inflatables.

11. The pericardial tissue transection device of claim 10, wherein the stabilizing members are located on an opposite side of the elongated body from the U-shaped notch.

12. The pericardial tissue transection device of claim 1, further comprising an atraumatic tip adjacent the distal end of the elongated body.

13. The pericardial tissue transection device of claim 1, wherein the elongated body comprises a lumen structured to receive a guidewire, such that the elongated body is slidable relative to the guidewire.

14. The pericardial tissue transection device of claim 1, wherein the surface of the elongated body comprises a tapered portion adjacent the wall surface of the U-shaped notch.

15. The pericardial tissue transection device of claim 1, wherein a distal end of the upper extension tapers to a blunt tip.

16. A pericardial tissue transection device comprising:
an elongated body extending along a longitudinal axis between proximal and distal ends, the elongated body comprising:
a U-shaped notch defined in the elongated body, wherein the U-shaped notch comprises a wall surface extending from a surface of the elongated body and an upper extension extending from the wall surface substantially parallel with the longitudinal axis of the elongated body, wherein the surface of the elongated body comprises a tapered portion adjacent the wall surface of the U-shaped notch to direct tissue into the U-shaped notch;
an incision member located in the U-shaped notch and extending from at least a portion of the wall surface of the U-shaped notch toward the extension of the U-shaped notch at an angle, wherein the incision member is structured to receive and to cut pericardial tissue passing through the U-shaped notch; and
stabilizing members structured to reversibly extend laterally from the elongated body, wherein the stabilizing members are located on an opposite side of the elongated body from the U-shaped notch.

17. A pericardial tissue transection device comprising:
an elongated body with proximal end and a distal end, and a longitudinal axis; and
a plurality of elongated members extending from a circumference of the distal end and coupled to a circumference of a coupling member, at least one of the plurality of elongated members comprising at least one incision portion,
a first configuration where the plurality of elongated members extend substantially parallel with the longitudinal axis, and
a second configuration where at least one of the plurality of elongated members comprising the at least one incision portion extend laterally from the elongated body.

18. The pericardial tissue transection device of claim 17, wherein the coupling member reversibly translatable from the distal end along the longitudinal axis causing the plurality of elongated members comprising the at least one incision portion to extend laterally.

19. The pericardial tissue transection device of claim 17, further comprising an actuator member extending from a center of the distal end and coupled to a center of the coupling member such that translation of the actuator member translates the coupling member proximally to the distal end of the elongated body causing the at least one incision portion to extend laterally beyond an outer diameter of the elongated body, the incision member configured to receive and to cut pericardial tissue. at least one of the plurality of elongated members extend beyond an outer diameter of the elongated body and engage pericardial tissue.

20. The pericardial tissue transection device of claim 17, wherein the incision portion extends laterally beyond an outer diameter of the elongated body and at least one of the plurality of elongated members extend beyond an outer diameter of the elongated body providing stabilization of the elongated body.
